(19) United States
(12) Patent Application Publication
(10) Pub. No.: US 2008/0293932 A1
(43) Pub. Date: Nov. 27, 2008

(54) PHTHALOCYANINE RECORDING LAYER OF OPTICAL RECORDING MEDIA

(75) Inventors: Fu-Shing Wang, Taoyuan (TW); Kuang-Mei Hsu, Taoyuan (TW)

Correspondence Address: KIRTON AND MCCONKIE, 60 EAST SOUTH TEMPLE,, SUITE 1800, SALT LAKE CITY, UT, 84111, US

(73) Assignee: TUBE SMITH TECHNOLOGY CO.,LTD., Taichung (TW)

(21) Appl. No.: 11/829,726

(22) Filed: Jul. 27, 2007

Related U.S. Application Data
(60) Provisional application No. 60/940,082, filed on May 25, 2007.

(51) Int. Cl. C07D 245/00; G01N 21/00

(52) U.S. Cl. 540/472; 436/164

(57) ABSTRACT

A phthalocyanine compound of formula (I) is provided for use in a recording layer of an optical recording media.

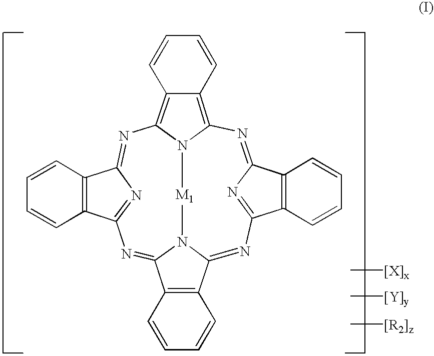

In formula (I),
M₁ is a divalent transition metal, or two hydrogen atoms;
X is halogen;
Y is —OR₁;
R₁ is alkyl which is unsubstituted or substituted by halogen, halogen, hydroxyl, alkoxy, alkylamino;
R₂ is

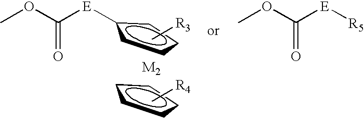

R₃ and R₄ independently denote H, halogen, alkyl and alkoxyl;
R₅ is unsubstituted or substituted phenyl, benzyl, nathalenyl, or heterocycle;
E denotes a single bond, C₁-C₄ alkyl, R₆—C—O—C(═O), R₆—O—C(═O)— or R₆—S—C(═O)—;
R₆ is unsubstituted or substituted phenyl, benzyl, nathalenyl, heterocycle or C₁-C₄ alkyl;
x is a number from 0 to 8;
y is a number from 0 to 8;
z is a number from 0 to 4; and
M₂ is a divalent transition metal.

### CROSS REFERENCE TO RELATED PATENT APPLICATION

This application claims the benefit of Provisional Patent Application Ser. No. 60/940,082 filed May 25, 2007.

### FIELD OF THE INVENTION

The present invention relates to a phthalocyanine compound, and more particularly to a phthalocyanine compound for use in a recording layer of an optical recording media.

### BACKGROUND OF THE INVENTION

Optical recording media such as the CD-R, DVD-R and the like have been widely used as recording media for recording digital data. These optical recording media, which are referred as write-once type optical recording media, enable writing but not rewriting of data.

In a write-once type optical recording medium, an organic dye such as a phthalocyanine dye is generally used as the material of the recording layer. By utilizing changes in an optical characteristic caused by chemical change of the phthalocyanine dye, data are recorded in the optical recording medium.

The properties of the phthalocyanine dye depend very much on metallization, substitution and crystal modification. Many improved structures of phthalocyanine dye have been disclosed for imparting one or more satisfactory properties to the recording layer of the optical recording medium. For example, the most stringent requirements include high refractive index, high reflectivity, uniformity of the writing width at different pulse duration, high light stability in daylight, high sensitivity to intense laser radiation, low noise, high resolution, very little statistical jitter of the pits over a desired value at optimum writing performance.

Since the recording layer is usually applied from a solution by spin-coating, the dyes should also be readily soluble in conventional solvents such as dibutylether or ethyl cyclohexane. However, unsubstituted phthalocyanine compounds are slightly soluble or insoluble in most solvents and therefore considerably lack workability. The slight solubility or insolubility in solvents of conventional phthalocyanine compounds is a great obstacle to the mass production of the recording layers of the write-once type optical recording media.

U.S. Pat. Nos. 6,087,492 and 6,399,768 have disclosed substituted phthalocyanine compounds for use as organic dyes of the recording layers of the write-once type optical recording media. These substituted phthalocyanine compounds include for example brominated tetra(2,4-dimethyl-3-pentyloxy)copper phthalocyanine and ester of brominated di(hydroxymethyl)tetra(2,4-dimethyl-3-pentyloxy)copper phthalocyanine and ferrocenecarboxylic acid. After the substituted phthalocyanine compounds are applied to CD-R discs, high reflectivity and very little jitter are achieved. The recording layers containing the substituted phthalocyanine compounds show very good recording properties at a lower speed (e.g. 1×-8×) but less favorable at a higher speed (e.g. 48×).

Moreover, the solubility in solvents for these substituted phthalocyanine compounds is still unsatisfactory and needs to be further improved.

### SUMMARY OF THE INVENTION

The present invention provides a phthalocyanine compound which is readily soluble in a solvent.

The present invention also provides a phthalocyanine compound for use in a recording layer of an optical recording media, which has very good recording properties at a high speed (e.g. 48×).

In an aspect, the present invention relates to a phthalocyanine compound of formula (I) for use in a recording layer of an optical recording media:

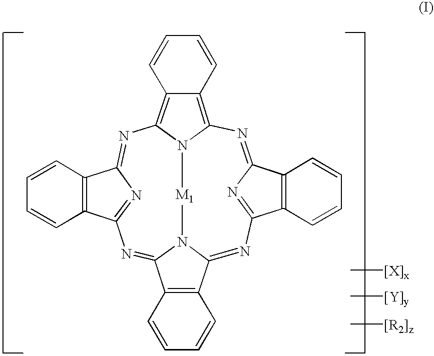

wherein
M₁ is a divalent transition metal, or two hydrogen atoms;
X is halogen;
Y is —OR₁;
R₁ is alkyl which is unsubstituted or substituted by halogen, halogen, hydroxyl, alkoxy, alkylamino;
R₂ is

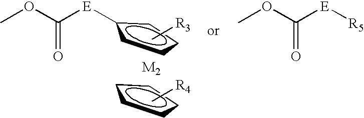

R₃ and R₄ independently denote H, halogen, alkyl and alkoxyl;
R₅ is unsubstituted or substituted phenyl, benzyl, nathalenyl, or heterocycle;
E denotes a single bond, C₁-C₄ alkyl, R₆—C—O—C(═O), R₆—O—C(═O)— or R₆—S—C(═O)—;
R₆ is unsubstituted or substituted phenyl, benzyl, nathalenyl, heterocycle or C₁-C₄ alkyl;
x is a number from 0 to 8;
y is a number from 0 to 8;
z is a number from 0 to 4; and
M₂ is a divalent transition metal.

In an embodiment, M₁ is H₂, or a divalent transition metal selected from a group consisting of Cu(II), Zn(II), Ni(II), Pd(II), Pt(II), Mn(II) and Co(II).

Preferably, M₁ is Cu(II) and M₂ is a Fe(II).

In an embodiment, E is R₆—C—O—C(═O), and R₆ is unsubstituted or substituted phenyl, benzyl, nathalenyl or heterocycle.

In an embodiment, E is benzyl-C—O—C(═O).

Preferably, the phthalocyanine compound corresponds to the formula (I-a),

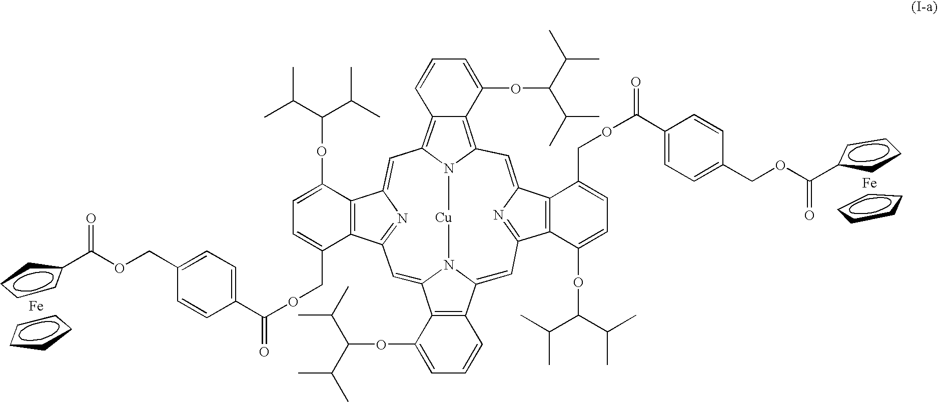

Preferably, E is a single bond, and R₅ is a substituted phenyl.

Preferably, the phthalocyanine compound corresponds to the formula (I-b),

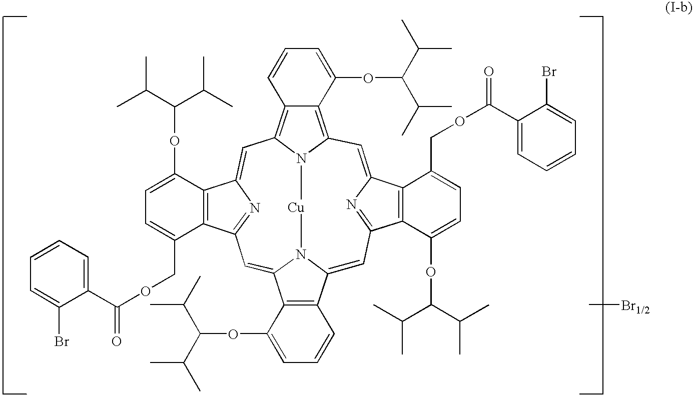

In another aspect, the present invention relates to a mixture of phthalocyanine compounds for use in a recording layer of an optical recording media. The mixture of phthalocyanine compounds comprises:
a first phthalocyanine compound of the formula (I-a),

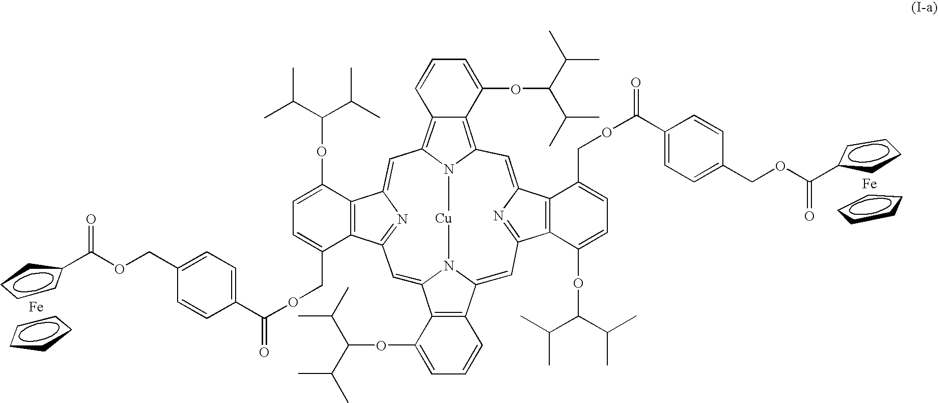

and
a second phthalocyanine compound of formula (I-b),

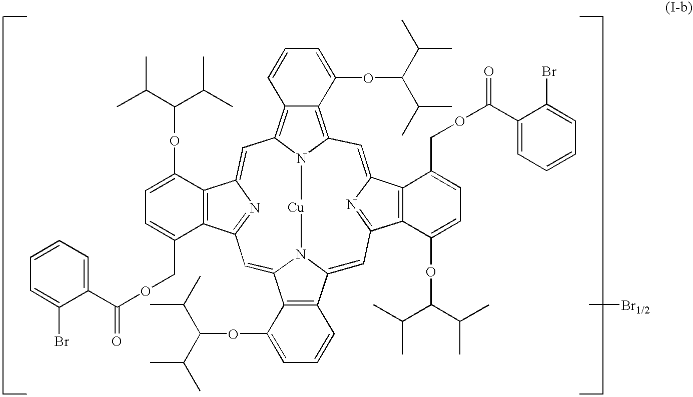

Preferably, the mixture comprises 20 to 80 wt % of the first phthalocyanine compound and 80 to 20 wt % of the second phthalocyanine compound, based on the total weight of the first and second phthalocyanine compounds.

More preferably, the mixture comprises 45 to 55 wt % of the first phthalocyanine compound and 55 to 45 wt % of the second phthalocyanine compound, based on the total weight of the first and second phthalocyanine compounds.

### DETAILED DESCRIPTION OF THE PREFERRED EMBODIMENT

The present invention will now be described more specifically with reference to the following embodiments. It is to be noted that the following descriptions of preferred embodiments of this invention are presented herein for purpose of illustration and description only; it is not intended to be exhaustive or to be limited to the precise form disclosed.

The present invention is hereinafter described by way of examples, but is not intended to be restricted to these examples. In these examples, the temperature is indicated in Centigrade and all percentages are by weight unless otherwise indicated. In addition, all materials or reagents are used as received without further purification unless otherwise specified.

### EXAMPLE 1

A 5 L round-bottom flask, equipped with a magnetic stirrer, thermometer, a reflux condenser, a dropping funnel and a nitrogen inlet, is charged with 800 g (777.6 mmol) of tetra-(α-2,4-dimethyl-3-pentyloxy)copper phthalocyanine in 2400 ml of chlorobenzene, and 576 g (4261.6 mmol) of N-methylformanilide and 380 g (2478.3 mmol) of phosphorus(III) oxychloride are then dropwisely added under 60° C. The reaction mixture is stirred for 8 hr at 60° C. Then the mixture is cooled to room temperature, and then 1200 ml of water is added. After phase separation, the organic layer is washed once with 10% of NaHCO_{3 (aq)} solution. Then, 800 g of silica gel is added to the green organic phase, stirred for 30 min, and filtered. The filtrate is washed with 3×1200 ml of chlorobenzene. The green solution is collected and concentrated by evaporation to 1200 ml, and then poured into 12 L of acetonitrile. The mixture is stirred for 30 min, and then the product is collected by filtration. The product is dried in an oven at 100° C. to give 763 g of green powder. This corresponds to 90.4% of the theoretical yield. The λₘₐₓ is 710 nm in DBE.

### EXAMPLE 2

3 L round-bottom flask, equipped with a magnetic stirrer, thermometer, a reflux condenser, a dropping funnel and a nitrogen inlet, is charged with 550 g (506.9 mmol) of diformyl tetra-(α-2,4-dimethyl-3-pentyloxy)copper phthalocyanine in 1375 ml of chlorobenzene and 550 ml of water, and 40 g (250.3 mmol) of bromine are then dropwisely added under 40° C. The reaction mixture is stirred for 1 hr at 40° C., and then the mixture is cooled to room temperature. After phase separation, the organic layer is washed once with 550 ml of water, and with 3% NaHSO_{3 (aq)} solution. Then, 550 g of silica gel is added to the green organic phase, stirred for 30 min, and filtered. The filtrate is washed with 3×1100 ml of chlorobenzene. The green solution is collected and concentrated by evaporation to 1100 ml, and then poured into 11 L of acetonitrile. The mixture is stirred for 30 min, and then collected the products by filtration. The product is dried in an oven at 100° C. to give 542 g of green powder. This corresponds to 95.1% of the theoretical yield. The λₘₐₓ is 711 nm in DBE.

### EXAMPLE 3

A 5 L round-bottom flask, equipped with a magnetic stirrer, thermometer, a reflux condenser, a dropping funnel and a nitrogen inlet, is charged with 350 g (311.4 mmol) of brominated diformyl tetra-(α-2,4-dimethyl-3-pentyloxy)copper phthalocyanine in 1400 ml of toluene and 350 ml of methanol, and cooled to 5° C. Then, 25 g (660.9 mmol) of sodium borohydride is added. The reaction mixture is stirred for 2 hr at room temperature. The reaction mixture is dropwisely added, with stirring, to 1575 ml of water. After phase separation, 350 g of silica gel is added to the organic layer. The green solution is stirred for 30 min, and then filtered. The filtrate is washed with 3×700 ml of toluene. The green solution is collected and concentrated by evaporation to 700 ml, and then poured into 7 L of acetonitrile. The mixture is stirred for 30 min, and then the product is collected by filtration. The product is dried in an oven at 100° C. to give 313 g of green powder. This corresponds to 89.1% of the theoretical yield. The λₘₐₓ is 717.5 nm in DBE.

### EXAMPLE 4

The procedures in example 3 are carried out except that brominated diformyl tetra-(α-2,4-dimethyl-3-pentyloxy)copper phthalocyanine is replaced by diformyl tetra-(α-2,4-dimethyl-3-pentyloxy)copper phthalocyanine to give green powder. This corresponds to 86.2% of the theoretical yield. The λₘₐₓ is 716 nm in DBE.

### EXAMPLE 5

A 2L round-bottom flask, equipped with a magnetic stirrer, a thermometer, a reflux condenser and a nitrogen inlet, is charged with 250 g (221.7 mmol) of brominated di-(hydroxymethyl)tetra-(α-2,4-dimethyl-3-pentyloxy)copper phthalocyanine in 750 ml of dichloromethane. Then, 120.5 g (599.4 mmol) of 2-bromobenzoic acid, 128.1 g (620.8 mmol) of N,N′-dicyclohexylcarbodiimide, and 25 g (204.6 mmol) of 4-di(methylamino)pyridine are added. The green solution is heated to reflux, with stirring, for 1 hr. After the reaction mixture is cooled to room temperature, 250 g of silica gel is added to the reaction mixture and then filtered. The filtrate is washed with 3×250 ml of dichloromethane. The green solution is collected and concentrated by evaporation to 750 ml, and then poured into 7.5 L of acetonitrile. The mixture is stirred for 30 min, and then the crude product is collected by filtration. The crude product is dried in an oven at 100° C. to give 318.6 g of green powder.

The crude product is dissolved in 1 L of toluene, and then 250 g of silica gel is added with stirring for 30 min. After filtration, the filtrate is washed with 3×750 ml of toluene. The green solution is concentrated by evaporation to 750 ml, and then poured into 7.5 L of acetonitrile. The precipitates are filtered and washed with 3×500 ml of acetonitrile, and dried in an oven at 100° C. to give 296.2 g of green powder. This corresponds to 89.4% of the theoretical yield. The λₘₐₓ is 713.5 nm in DBE.

### EXAMPLE 6

The procedures in example 5 are carried out except that brominated diformyl tetra-(α-2,4-dimethyl-3-pentyloxy)copper phthalocyanine is replaced by diformyl tetra-(α-2,4-dimethyl-3-pentyloxy)copper phthalocyanine to give a green powder. This corresponds to 86.7% of the theoretical yield. The λₘₐₓ is 711 nm in DBE.

### EXAMPLE 7

The procedures in example 5 are carried out except that 2-bromobenzoic acid is replaced by 1-naphthoic acid to give green powder. This corresponds to 84.3% of the theoretical yield. The λₘₐₓ is 713 nm in DBE.

### EXAMPLE 8

The procedures in example 5 are carried out except that brominated diformyl tetra-(α-2,4-dimethyl-3-pentyloxy)copper phthalocyanine is replaced by diformyl tetra-(α-2,4-dimethyl-3-pentyloxy)copper phthalocyanine and 2-bromobenzoic acid is replaced by 1-naphthoic acid to give a green powder. This corresponds to 81.2% of the theoretical yield. The λₘₐₓ is 710.5 nm in DBE.

### EXAMPLE 9

The procedures in example 5 are carried out except that 2-bromobenzoic acid is replaced by 4-tert-butylbenzoic acid to give a green powder. This corresponds to 91.2% of the theoretical yield. The λₘₐₓ is 713 nm in DBE.

### EXAMPLE 10

The procedures in example 5 are carried out except that brominated diformyl tetra-(α-2,4-dimethyl-3-pentyloxy)copper phthalocyanine is replaced by diformyl tetra-(α-2,4-dimethyl-3-pentyloxy)copper phthalocyanine and 2-bromobenzoic acid is replaced by 4-tert-butylbenzoic acid to give a green powder. This corresponds to 89.5% of the theoretical yield. The λₘₐₓ is 711 nm in DBE.

### EXAMPLE 11

The procedures in example 5 are carried out except that 2-bromobenzoic acid is replaced by biphenyl carboxylic acid to give a green powder. This corresponds to 83.2% of the theoretical yield. The λₘₐₓ is 713.5 nm in DBE.

### EXAMPLE 12

The procedures in example 5 are carried out except that brominated diformyl tetra-(α-2,4-dimethyl-3-pentyloxy)copper phthalocyanine is replaced by diformyl tetra(α-2,4-dimethyl-3-pentyloxy)copper phthalocyanine and 2-bromobenzoic acid is replaced by biphenyl carboxylic acid to give a green powder. This corresponds to 83.6% of the theoretical yield. The λₘₐₓ is 711 nm in DBE.

### EXAMPLE 13

The procedures in example 5 are carried out except that 2-bromobenzoic acid is replaced by ferrocene carboxylic acid to give a green powder. This corresponds to 90.6% of the theoretical yield. The λₘₐₓ is 713.5 nm in DBE.

### EXAMPLE 14

The procedures in example 5 are carried out except that brominated diformyl tetra-(α-2,4-dimethyl-3-pentyloxy)copper phthalocyanine is replaced by diformyl tetra(α-2,4-dimethyl-3-pentyloxy)copper phthalocyanine and 2-bromobenzoic acid is replaced by ferrocene carboxylic acid to give a green powder. This corresponds to 89.8% of the theoretical yield. The λₘₐₓ is 710.5 nm in DBE.

### EXAMPLE 15

The procedures in example 5 are carried out except that 2-bromobenzoic acid is replaced by 4-formylbenzoic acid to give a green powder. This corresponds to 83.6% of the theoretical yield. The λₘₐₓ is 713 nm in DBE.

### EXAMPLE 16

The procedures in example 5 are carried out except that brominated diformyl tetra-(α-2,4-dimethyl-3-pentyloxy)copper phthalocyanine is replaced by diformyl tetra-(α-2,4-dimethyl-3-pentyloxy)copper phthalocyanine and 2-bromobenzoic acid is replaced by 4-formylbenzoic acid to give a green powder. This corresponds to 85.2% of the theoretical yield. The λₘₐₓ is 710 nm in DBE.

### EXAMPLE 17

A 2 L round-bottom flask, equipped with a magnetic stirrer, a thermometer, a reflux condenser, a dropping funnel and a nitrogen inlet, is charged with 250 g (179.5 mmol) of the compound of example 15 in 1000 ml of toluene and 250 ml of methanol, and cooled to 5° C. Then, 14.9 g (394.9 mmol) of sodium borohydride is added. The reaction mixture is stirred for 2 hr at room temperature. The reaction mixture is dropwisely added, with stirring, to 1125 ml of water. After phase separation, 250 g of silica gel is added to the organic layer. The green solution is stirred for 30 min, and then filtered. The filtrate is washed with 3×500 ml of toluene. The green solution is collected and concentrated by evaporation to 500 ml, and then poured into 5 L of acetonitrile. The mixture is stirred for 30 min, and then the product is collected by filtration. The product is dried in an oven at 100° C. to give 236 g of green powder. This corresponds to 94.2% of the theoretical yield. The λₘₐₓ is 714 nm in DBE.

### EXAMPLE 18

The procedures in example 17 are carried out except that the compound of example 15 is replaced by the compound of example 16. This corresponds to 92.4% of the theoretical yield. The λₘₐₓ is 710.5 nm in DB.

### EXAMPLE 19

A 2L round-bottom flask, equipped with a magnetic stirrer, a thermometer, a reflux condenser and a nitrogen inlet, is charged with 200 g (143.2 mmol) of the product prepared in preparation example 17 in 600 ml of dichloromethane. Then, 82.5 g (358.7 mmol) of ferrocene carboxylic acid, 77 g (373.2 mmol) of N,N′-dicyclohexylcarbodiimide, and 20 g (163.7 mmol) of 4-di(methylamino)pyridine are added and the green solution is heated to reflux, with stirring, for 30 min. After the reaction mixture is cooled to room temperature, 200 g of silica gel is added and filtered. The filtrate is washed with 3×200 ml of dichloromethane. The green solution is collected and concentrated by evaporation to 600 ml, and then poured into 6 L of acetonitrile. The mixture is stirred for 30 min, and then the crude product is collected by filtration. The crude product is dried in oven at 100° C. to give 232 g of green powder.

The crude product is dissolved in 1 L of toluene and 200 g of silica gel is added with stirring for 30 min. After filtration, the filtrate is washed with 3×600 ml of toluene. The green solution is concentrated by evaporation to 600 ml, and then poured into 6 L of acetonitrile. The precipitates is filtered and washed with 3×400 ml of acetonitrile, and then dried in an oven at 100° C. to give 224.3 g of green powder. This corresponds to 86% of the theoretical yield. The λₘₐₓ is 713.5 nm in DBE.

### EXAMPLE 20

The procedures in example 19 are carried out except that the compound of example 17 is replaced by the compound of example 18. This corresponds to 83.5% of the theoretical yield. The λₘₐₓ is 711 nm in DBE.

A 1.8% by weight solution of the compound of example 20 corresponding to formula I-b and compound of example 5 corresponding to formula I-a (at a weight ratio of 60:40) in a mixture consisting of ethyl cyclohexane is filtered through a Teflon filter having a pore width of 0.2 μm and is applied to the disc by spin-coating, and is written by LITE-ON LTR-48246S VSS06/48X. Triplicate test runs were conducted, and the results are shown in Table 1 and Table 2.

In these tables, the parameter BLER (BLock Error Rate) indicates the number of errors that occur per second at the first level of error correction. I3R and I11R measurements make it possible to distinguish between amplitude variations caused by pit deformation and amplitude variations caused by absorption in substrate and reflective layer. REF indicates the reflectivity of the tested disc. Jitter is measured individually for pit and land (3T, 11T). Beta measurements show as a percentage the difference between pits and lands on the disc. WAWM denotes the weighted average window margin jitter.

TABLE 1

| | BLER | | I3R | I11R | REF | Jitter | | | |
|---|---|---|---|---|---|---|---|---|---|
| | max | ave | min | Min | ave | land-3T | pit-3T | land-11T | pit-11T |
| Item | ≦50 | ≦10 | ≧0.3 | ≧0.6 | ≧60 | ≦35 | ≦35 | ≦35 | ≦35 |
| Disc 1 | 22 | 3.1 | 0.35 | 0.62 | 64.7 | 25 | 24 | 24 | 22 |
| Disc 2 | 21 | 2.9 | 0.35 | 0.62 | 64.9 | 24 | 24 | 23 | 22 |
| Disc 3 | 24 | 2.7 | 0.36 | 0.64 | 65.0 | 24 | 23 | 24 | 22 |

TABLE 2

| | Beta | | | Cu | WAWM | | |
|---|---|---|---|---|---|---|---|
| Item | min | max | ave | ave | min | max | ave |
| | −8~+12 | | | 0 | | | |
| Disc 1 | −11.4 | −2.0 | −6.71 | 0 | 35.95 | 46.04 | 39.637 |
| Disc 2 | −10.7 | −1.8 | −6.74 | 0 | 36.52 | 47.83 | 41.195 |
| Disc 3 | −10.7 | −1.5 | −6.55 | 0 | 36.93 | 48.56 | 41.169 |

These results shown in Table 1 and Table 2 demonstrate that the phthalocyanine dye of the present invention has very good recording properties at a high speed (e.g. 48×). Moreover, experiments show that the phthalocyanine dye of the present invention is readily soluble in conventional solvents such as dibutylether or ethyl cyclohexane.

While the invention has been described in terms of what is presently considered to be the most practical and preferred embodiments, it is to be understood that the invention needs not to be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A phthalocyanine compound of formula (I) for use in a recording layer of an optical recording media: 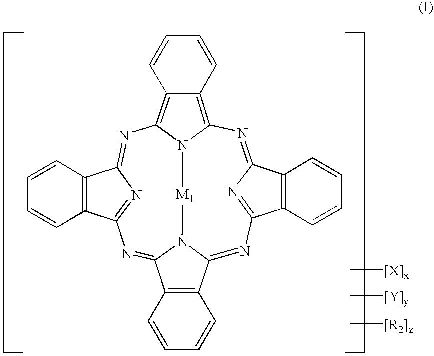
wherein
M₁ is a divalent transition metal, or two hydrogen atoms;
X is halogen;
Y is —OR₁;
R₁ is alkyl which is unsubstituted or substituted by halogen, halogen, hydroxyl, alkoxy, alkylamino;
R₂ is 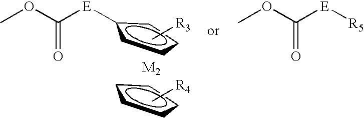
R₃ and R₄ independently denote H, halogen, alkyl and alkoxyl;
R₅ is unsubstituted or substituted phenyl, benzyl, nathalenyl, or heterocycle;
E denotes a single bond, C₁-C₄ alkyl, R₆—C—O—C(═O), R₆—O—C(═O)— or R₆—S—C(═O)—;
R₆ is unsubstituted or substituted phenyl, benzyl, nathalenyl, heterocycle or C₁-C₄ alkyl;
x is a number from 0 to 8;
y is a number from 0 to 8;
z is a number from 0 to 4; and
M₂ is a divalent transition metal.

2. The phthalocyanine compound according to claim 1 wherein M₁ is H₂, or a divalent transition metal selected from a group consisting of Cu(II), Zn(II), Ni(II), Pd(II), Pt(II), Mn(II) and Co(II).

3. The phthalocyanine compound according to claim 2 wherein M₁ is Cu(II).

4. The phthalocyanine compound according to claim 1 wherein M₂ is a Fe(II).

5. The phthalocyanine compound according to claim 1 wherein E is R₆—C—O—C(═O), and R₆ is unsubstituted or substituted phenyl, benzyl, nathalenyl, heterocycle or C₁-C₄ alkyl.

6. The phthalocyanine compound according to claim 5 wherein E is benzyl-C—O—C(═O).

7. The phthalocyanine compound according to claim 6 wherein the phthalocyanine compound corresponds to the formula (I-a),

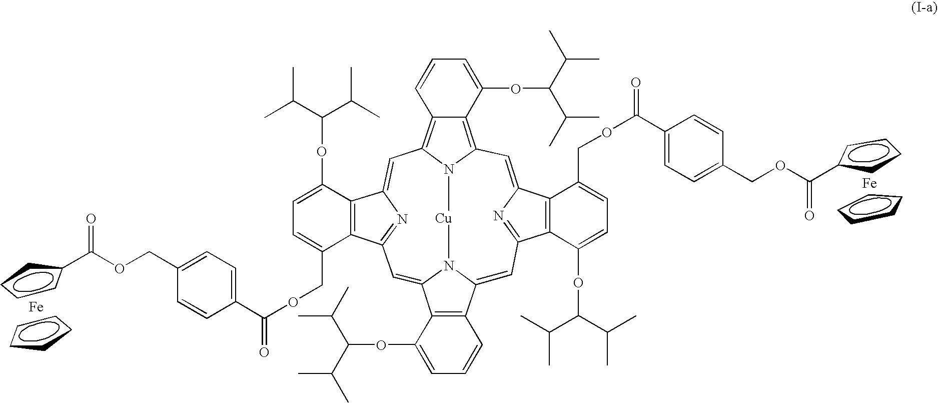

8. The phthalocyanine compound according to claim 1 wherein E is a single bond, and R₅ is a substituted phenyl.

9. The phthalocyanine compound according to claim 8 wherein the phthalocyanine compound corresponds to the formula (I-b),

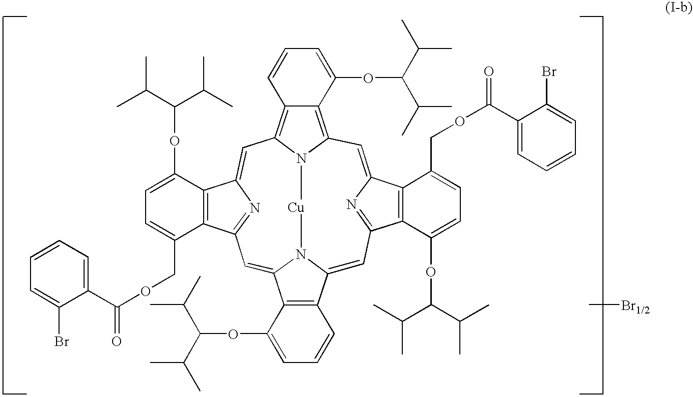

10. A mixture of phthalocyanine compounds for use in a recording layer of an optical recording media, comprising:
a first phthalocyanine compound of the formula (I-a), 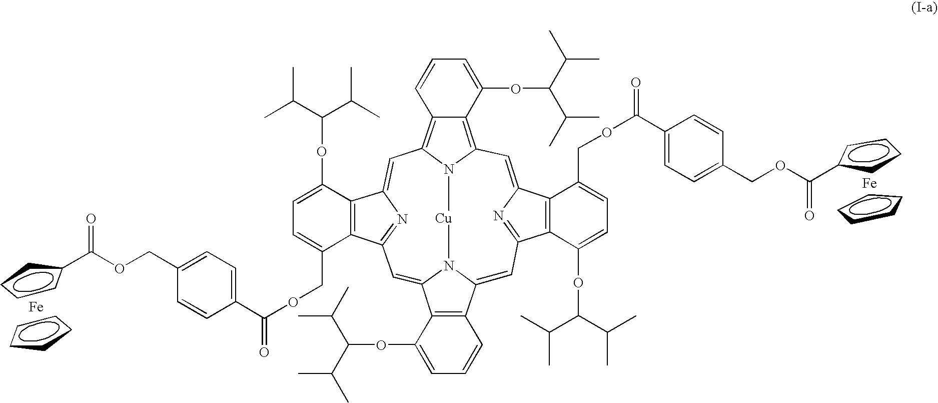
and
a second phthalocyanine compound of formula (I-b),

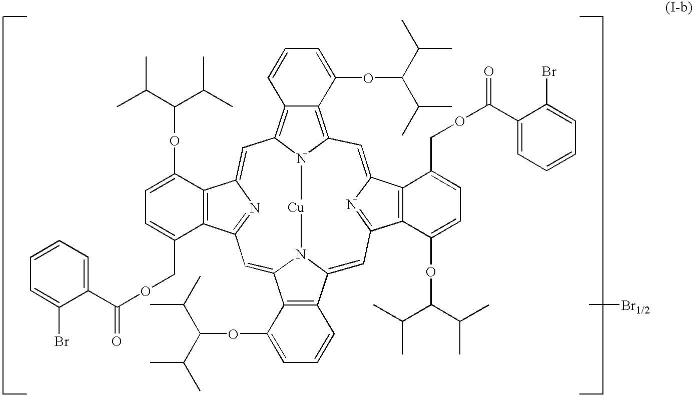

11. The mixture according to claim 10, comprising 20 to 80 wt % of the first phthalocyanine compound and 80 to 20 wt % of the second phthalocyanine compound, based on the total weight of the first and second phthalocyanine compounds.

12. The mixture according to claim 10, comprising 45 to 55 wt % of the first phthalocyanine compound and 55 to 45 wt % of the second phthalocyanine compound, based on the total weight of the first and second phthalocyanine compounds.
